(19) United States
(12) Patent Application Publication
(10) Pub. No.: US 2006/0127440 A1
(43) Pub. Date: Jun. 15, 2006

(54) Drug delivery systems for the prevention and treatment of vascular diseases

(76) Inventor: Margaret Forney Prescott, Millburn, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/352,767

(22) Filed: Feb. 13, 2006

Related U.S. Application Data
(63) Continuation of application No. 10/339,820, filed on Jan. 10, 2003, now abandoned.
(60) Provisional application No. 60/347,264, filed on Jan. 10, 2002.

(51) Int. Cl. A61K 31/4745; A61F 2/06

(52) U.S. Cl. 424/422; 514/291; 623/1.11

(57) ABSTRACT

Provided are drug delivery systems for the prevention and treatment of proliferative diseases, particularly vascular diseases, comprising rapamycin or a rapamycin derivative having mTOR inhibiting properties, optionally in conjunction with one or more active co-agents.

This application claims the benefit of Provisional Application No. 60/347,264, filed Jan. 10, 2002, which in its entirety is herein incorporated by reference.

### FIELD OF INVENTION

The present invention relates to drug delivery systems for the prevention and treatment of proliferative diseases, particularly vascular diseases.

### BACKGROUND OF THE INVENTION

Many humans suffer from circulatory diseases caused by a progressive blockage of the blood vessels that perfuse the heart and other major organs. Severe blockage of blood vessels in such humans often leads to ischemic injury, hypertension, stroke or myocardial infarction. Atherosclerotic lesions which limit or obstruct coronary or periphery blood flow are the major cause of ischemic disease related morbidity and mortality including coronary heart disease and stroke. To stop the disease process and prevent the more advanced disease states in which the cardiac muscle or other organs are compromised, medical revascularization procedures such as percutaneous transluminal coronary angioplasty (PCTA), percutaneous transluminal angioplasty (PTA), atherectomy, bypass grafting or other types of vascular grafting procedures are used.

Re-narrowing (restenosis) of an artherosclerotic coronary artery after various revascularization procedures occurs in 10-80% of patients undergoing this treatment, depending on the procedure used and the aterial site. Besides opening an artery obstructed by atherosclerosis, revascularization also injures endothelial cells and smooth muscle cells within the vessel wall, thus initiating a thrombotic and inflammatory response. Cell derived growth factors such as platelet derived growth factor, infiltrating macrophages, leukocytes or the smooth muscle cells themselves provoke proliferative and migratory responses in the smooth muscle cells. Simultaneous with local proliferation and migration, inflammatory cells also invade the site of vascular injury and may migrate to the deeper layers of the vessel wall. Proliferation/migration usually begins within one to two days post-injury and, depending on the revascularization procedure used, continues for days and weeks.

Both cells within the atherosclerotic lesion and those within the media migrate, proliferate and/or secrete significant amounts of extracellular matrix proteins. Proliferation, migration and extracellular matrix synthesis continue until the damaged endothelial layer is repaired at which time proliferation slows within the intima. The newly formed tissue is called neointima, intimal thickening or restenotic lesion and usually results in narrowing of the vessel lumen. Further lumen narrowing may take place due to constructive remodeling, e.g. vascular remodeling, leading to further intimal thickening or hyperplasia.

Furthermore, there are also atherosclerotic lesions which do not limit or obstruct vessel blood flow but which form the so-called “vulnerable plaques”. Such atherosclerotic lesions or vulnerable plaques are prone to rupture or ulcerate, which results in thrombosis and thus produces unstable angina pectoris, myocardial infarction or sudden death. Inflamed atherosclerotic plaques can be detected by thermography.

Alternatively, complications associated with vascular access treatment is a major cause of morbidity in many disease states. For example, vascular access dysfunction in hemodialysis patients is generally caused by outflow stenoses in the venous circulation (Schwam S. J., et al., Kidney Int. 36: 707-711, 1989). Vascular access related morbidity accounts for about 23 percent of all hospital stays for advanced renal disease patients and contributes to as much as half of all hospitalization costs for such patients (Feldman H. I., J. Am. Soc. Nephrol. 7: 523-535, 1996).

Additionally, vascular access dysfunction in chemotherapy patients is generally caused by outflow stenoses in the venous circulation and results in a decreased ability to administer medications to cancer patients. Often the outflow stenoses is so severe as to require intervention.

Additionally, vascular access dysfunction in total parenteral nutrition (TPN) patients is generally caused by outflow stenoses in the venous circulation and results in reduced ability to care for these patients.

Up to the present time, there has not been any effective drug for the prevention or reduction of vascular access dysfunction in association with the insertion or repair of an indwelling shunt, fistula or catheter, preferably a large bore catheter, into a vein in a mammal, particularly a human patient.

Survival of patients with chronic renal failure depends on optimal regular performance of dialysis. If this is not possible (for example as a result of vascular access dysfunction or failure), it leads to rapid clinical deterioration and unless the situation is remedied, these patients will die. Hemodialysis requires access to the circulation. The ideal form of hemodialysis vascular access should allow repeated access to the circulation, provide high blood flow rates, and be associated with minimal complications. At present, the three forms of vascular access are native arteriovenous fistulas (AVF), synthetic grafts, and central venous catheters. Grafts are most commonly composed of polytetrafluoroethylene (PTFE) or Gore-Tex. Each type of access has its own advantages and disadvantages.

Vascular access dysfunction is the most important cause of morbidity and hospitalization in the hemodialysis population. Venous neointimal hyperplasia characterized by stenosis and subsequent thrombosis accounts for the overwhelming majority of pathology resulting in dialysis graft failure. The most common form of vascular access procedure performed in chronic hemodialysis patients in the United States is the arteriovenous PTFE graft, which accounts for approximately 70% of all hemodialysis access.

Dr. Burnett S. Kelly and Col., (Kidney International, Volume 62; Issue 6; Page 2272—December 2002) and others have previously shown that venous neointimal hyperplasia (VNH) in the setting of arteriovenous hemodialysis grafts is characterized by smooth muscle cells, neointimal and adventitial microvessels and extracellular matrix components. However, despite a reasonable knowledge of the pathology of VNH, there are still no effective interventions for either the prevention or treatment of hemodialysis vascular access dysfunction. This is particularly unfortunate, as VNH in the setting of hemodialysis grafts appears to be a far more aggressive lesion as compared to the more common arterial neointimal hyperplasia that occurs in peripheral bypass grafts. Compare the 50% one year primary patency in PTFE dialysis access grafts with an 88% five year patency for aortoiliac grafts and a 70 to 80% one year patency for femoro-popliteal grafts. Venous stenoses in the setting of dialysis access grafts also have a poorer response to angioplasty (40% three month survival if thrombosed and a 50% six month survival if not thrombosed) as compared to arterial stenoses. They believe that the lack of effective therapies for VNH and venous stenosis in dialysis grafts such as PTFE dialysis grafts is due to (a) a lack of appreciation of the fact that venous stenosis may be very different from the more common arterial stenosis at the graft-artery anastomosis and (b) the absence of a validated large animal model of VNH to test out novel interventions.

Despite the magnitude of the problem and the enormity of the cost, there are currently no effective therapies for the prevention or treatment of venous neointimal hyperplasia in dialysis grafts.

Accordingly, there is a need for effective treatment and drug delivery systems for revascularization procedure, e.g. preventing and treating intimal thickening or restenosis that occurs after injury, e.g. vascular injury, including e.g. surgical injury, e.g. revascularization-induced injury, e.g. also in heart or other grafts, for a stabilization procedure of vulnerable plaques, or for the prevention or treatment of vascular access dysfunctions.

### SUMMARY OF THE INVENTION

It has now been found that rapamycin and rapamycin derivatives having mTOR inhibiting properties, optionally in conjunction with other active compounds, e.g. antiproliferative compounds, have beneficial effects on above mentioned disorders, diseases or dysfunctions.

### DETAILED DESCRIPTION OF THE INVENTION

Rapamycin is a known macrolide antibiotic produced by Streptomyces hygroscopicus, which inhibits mTOR. By rapamycin derivative having mTOR inhibiting properties is meant a substituted rapamycin, e.g. a 40-substituted-rapamycin or a 16-substituted rapamycin, or a 32-hydrogenated rapamycin, for example a compound of formula I 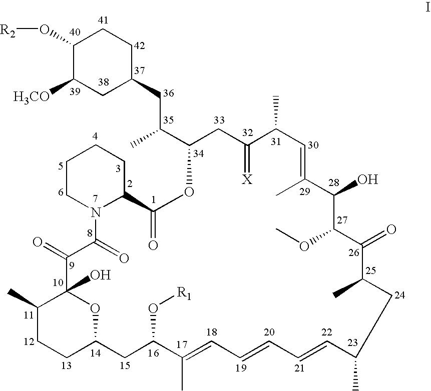
wherein
R₁ is CH₃ or C₃₋₆alkynyl,
R₂ is H, —CH₂—CH₂—OH, 3-hydroxy-2-(hydroxymethyl)-2-methyl-propanoyl or tetrazolyl, and
X is ═O, (H,H) or (H,OH)
   provided that R₂ is other than H when X is ═O and R₁ is CH₃,
   or a prodrug thereof when R₂ is —CH₂—CH₂—OH, e.g. a physiologically hydrolysable ether thereof.

Representative rapamycin derivatives of formula I are e.g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779) or 40-epi-(tetrazolyl)-rapamycin (also called ABT578). A preferred compound is e.g. 40-0-(2-hydroxyethyl)-rapamycin disclosed in Example 8 in WO 94/09010, or 32-deoxorapamycin or 16-pent-2-ynyloxy-32(S)-dihydro-rapamycin as disclosed in WO 96/41807.

Rapamycin derivatives may also include the so-called rapalogs, e.g. as disclosed in WO 98/02441 and WO01/14387, e.g. AP23573.

According to the invention, rapamycin or a rapamycin derivative having mTOR inhibiting properties may be applied as the sole active ingredient or in conjunction with one or more active co-agents selected from
a) an immunosuppressive agent, e.g. a calcineurin inhibitor, e.g. a cyclosporin, for example cyclosporin A, ISA tx 247 or FK506,
b) an EDG-receptor agonist having lymphocyte depleting properties, e.g. FTY720 (2-amino-2-[2-(4-octylphenyl) ethyl]propane-1,3-diol in free form or in a pharmaceutically acceptable salt form, e.g. the hydrochloride) or an analogue such as described in WO96/06068 or WO 98/45249, e.g. 2-amino-2-{2-[4-(1-oxo-5-phenylpentyl)phenyl]ethyl}propane-1,3-diol or 2-amino-4-(4-heptyloxyphenyl)-2-methyl-butanol in free form or in a pharmaceutically acceptable salt form,
c) an anti-inflammatory agent, e.g. a steroid, e.g. a corticosteroid, e.g. dexamethasone or prednisone, a NSAID, e.g. a cyclooxygenase inhibitor, e.g. a cox-2 inhibitor, e.g. celecoxib, rofecoxib, etoricoxib or valdecoxib, an ascomycin, e.g. ASM981 (or pimecrolimus), a cytokine inhibitor, e.g. a lymphokine inhibitor, e.g. an IL-1, -2 or -6 inhibitor, for example pralnacasan or anakinra, or a TNF inhibitor, for instance Etanercept, or a chemokine inhibitor;
d) an anti-thrombotic or anti-coagulant agent, e.g. heparin or a glycoprotein IIb/IIIa inhibitor, e.g. abciximab, eptifibatide or tirofibran;
e) an antiproliferative agent, e.g.
a microtubule stabilizing or destabilizing agent including but not limited to taxanes, e.g. taxol, paclitaxel or docetaxel, vinca alkaloids, e.g. vinblastine, especially vinblastine sulfate, vincristine especially vincristine sulfate, and vinorelbine, discodermolides or epothilones or a derivative thereof,e.g. epothilone B or a derivative thereof;
a protein tyrosine kinase inhibitor, e.g. protein kinase C or PI(3) kinase inhibitor, for example staurosporin and related small molecules, e.g. UCN-01, BAY 43-9006, Bryostatin 1, Perifosine, Limofosine, midostaurin, CGP52421, RO318220, RO320432, GO 6976, Isis 3521, LY333531, LY379196, SU5416, SU6668, AG1296, imatinib, etc.;
a compound or antibody which inhibits the PDGF receptor tyrosine kinase or a compound which binds to PDGF or reduces expression of the PDGF receptor e.g. a N-phenyl-2-pyrimidine-amine derivative, e.g. imatinib, CT52923, RP-1776, GFB-111, a pyrrolo[3,4-c]-beta-carboline-dione, etc.;
a compound or antibody which inhibits the EGF receptor tyrosine kinase or a compound which binds to EGF or reduces expression of the EGF receptor e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839, Iressa) and WO 95/03283 (e.g. compound ZM105180); e.g. trastuzumab (Herpetin®), cetuximab, OSI-774, CI-1033, EKB-569, GW-2016, E1.1, E2.4, E2.5, E6.2, E6.4, E2.11, E6.3 or E7.6.3, retinoic acid, alpha-, gamma- or delta-tocopherol or alpha-, gamma- or delta-tocotrienol, or compounds affecting GRB2, IMC-C225; or
a compound or antibody which inhibits the VEGF receptor tyrosine kinase or a VEGF receptor or a compound which binds to VEGF, e.g. proteins, small molecules or monoclonal antibodies generically and specifically disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819, WO 00/37502, WO 94/10202 and EP 0 769 947, those as described by M. Prewett et al in Cancer Research 59 (1999) 5209-5218, by F. Yuan et al in Proc. Natl. Acad. Sci. USA, vol. 93, pp. 14765-14770, Dec. 1996, by Z. Zhu et al in Cancer Res. 58, 1998, 3209-3214, by J. Mordenti et al in Toxicologic Pathology, Vol. 27, no. 1, pp 14-21, 1999, Angiostatin™, described by M. S. O'Reilly et al, Cell 79, 1994, 315-328, Endostatin™, described by M. S. O'Reilly et al, Cell 88, 1997, 277-285, anthranilic acid amides, ZD4190; ZD6474, SU5416, SU6668 or anti-VEGF antibodies or anti-VEGF receptor antibodies, e.g. RhuMab;
f) a statin, e.g. having HMG-CoA reductase inhibition activity, e.g. fluvastatin, lovastatin, simvastatin, pravastatin, atorvastatin, cerivastatin, pitavastatin, rosuvastatin or nivastatin;
g) a compound, protein, growth factor or compound stimulating growth factor production that will enhance endothelial regrowth of the luminal endothelium, e.g. FGF, IGF;
h) a matrix metalloproteinase inhibitor, e.g. batimistat, marimistat, trocade, CGS 27023, RS 130830 or AG3340;
k) a modulator (i.e. antagonists or agonists) of kinases, e.g. JNK, ERK1/2, MAPK or STAT;
l) a compound stimulating the release of (NO) or a NO donor, e.g. diazeniumdiolates, S-nitrosothiols, mesoionic oxatriazoles, isosorbide or a combination thereof, e.g. mononitrate and/or dinitrate;
m) a somatostatin analogue, e.g. octreotide, lanreotide, vapreotide or a cyclohexapeptide having somatostatin agonist properties, e.g. cyclo[4-(NH₂—C₂H₄—NH—CO—O)Pro-Phg-DTrp-Lys-Tyr(Bzl)-Phe]; or a modified GH analogue chemically linked to PEG, e.g. Pegvisomant;
n) an altosterone synthetase inhibitor or aldosterone receptor blocker, e.g. eplerenone, or a compound inhibiting the renin-angiotensin system, e.g. a renin inhibitor, e.g. SPP100, an ACE inhibitor, e.g. captopril, enalapril, lisinopril, fosinopril, benazepril, quinapril, ramipril, imidapril, perindopril erbumine, trandolapril or moexipril, or an ACE receptor blocker, e.g. losartan, irbesartan, candesartan cilexetil, valsartan or olmesartan medoxomil;
o) mycophenolic acid or a salt thereof, e.g. sodium mycophenolate, or a prodrug thereof, e.g. mycophenolate mofetil.

Are comprised also in the above list the pharmaceutically acceptable salts, the corresponding racemates, diastereoisomers, enantiomers, tautomers as well as the corresponding crystal modifications of above disclosed compounds where present, e.g. solvates, hydrates and polymorphs.

By antibody is meant monoclonal antibodies, polyclonal antibodies, multispecific antibodies formed from at least 2 intact antibodies, and antibodies fragments so long as they exhibit the desired biological activity.

A pharmaceutical combination comprising i) rapamycin or a rapamycin derivative having mTOR properties and ii) pimecrolimus, also form part of the present invention.

According to the invention, rapamycin is preferably locally administered or delivered in conjunction with one or more co-agents selected from b), e), f), g), h), k), m), n), o), a cox-2 inhibitor, a cytokine inhibitor or a chemokine inhibitor, as defined above.

In accordance with the particular findings of the present invention, there is provided
1.1 A method for preventing or treating smooth muscle cell proliferation and migration in hollow tubes, or increased cell proliferation or decreased apoptosis or increased matrix deposition in a subject in need thereof, comprising local administration of a therapeutically effective amount of rapamycin or a rapamycin derivative having mTOR inhibiting properties, optionally in conjunction with one or more other active co-agents, e.g. as disclosed above.
1.2 A method for the prevention or treatment of intimal thickening in vessel walls comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
Preferably the intimal thickening in vessel walls is stenosis, restenosis, e.g. following revascularization or neovascularization, and/or inflammation and/or thrombosis.
1.3 A method for the prevention or treatment of inflammatory disorders, e.g. T-cell induced inflammation, in hollow tubes comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
1.4 A method for stabilizing vulnerable plaques in blood vessels of a subject in need of such a stabilization comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
1.5 A method as defined in 1.1 to 1.4 associated, simultaneously or sequentially, with the administration of a therapeutically effective amount of rapamycin or a derivative thereof having mTOR inhibiting properties, e.g. a compound of formula I. Preferably rapamycin or the derivative thereof, e.g. of formula I, is administered orally.
Alternatively, a method as defined in 1.1 to 1.4 may be associated, simultaneously or sequentially, with the administration of a therapeutically effective amount of the co-agent.
1.6 A method for preventing or treating restenosis in diabetic patients comprising administering to said patients a therapeutically effective amount of rapamycin or a rapamycin derivative having mTOR inhibiting properties, optionally in conjunction with one or more other active co-agents, e.g. as disclosed above.
1.7 A method for preventing or treating restenosis in diabetic patients comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
1.8 A method comprising a combination of method steps as disclosed above under 1.6 and 1.7.
1.9 A method for the prevention or reduction of vascular access dysfunction in association with the insertion or repair of an indwelling shunt, fistula or catheter, preferably a large bore catheter, into a vein or artery, or actual treatment, in a subject in need thereof, which comprises administering to the subject rapamycin or a rapamycin derivative having mTOR inhibiting properties, optionally in conjunction with one or more other active co-agents, e.g. as disclosed above, or a controlled delivery from a drug delivery medical device or system of a therapeutically effective amount of rapamycin or a rapamycin derivative having mTOR inhibiting properties, optionally in conjunction with one or more other active co-agents, e.g. as disclosed above.
Preferably the invention relates to the prevention or reduction of vascular access dysfunction in hemodialysis.
1.10 A method for the stabilization or repair of arterial or venous aneurisms in a subject comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
1.11 A method for the prevention or treatment of anastomic hyperplasia in a subject comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
1.12 A method for the prevention or treatment of arterial, e.g. aortic, by-pass anastomosis in a subject comprising the controlled delivery from any catheter-based device, intraluminal medical device or adventitial medical device of a therapeutically effective amount of rapamycin or a rapamycin derivative having mTOR inhibiting properties, optionally in conjunction with one or more other active co-agents, e.g. as disclosed above.
1.13 A method as defined in 1.9 to 1.12 associated, simultaneously or sequentially, with the administration of a therapeutically effective amount of rapamycin or a derivative thereof, e.g. a compound of formula I. Preferably rapamycin or the derivative thereof, e.g. of formula I, is administered orally.
Alternatively, a method as defined in 1.9 to 1.12 may be associated, simultaneously or sequentially, with the administration of a therapeutically effective amount of the co-agent.
2.1 A drug delivery device or system comprising i) a medical device adapted for local application or administration in hollow tubes, e.g. a catheter-based delivery device or a medical device intraluminal or outside of hollow tubes such as an implant or a sheath placed within the adventitia, and ii) a therapeutic dosage of a rapamycin derivative having mTOR inhibiting properties or rapamycin, optionally in conjunction with a therapeutic dosage of one or more other active co-agents, e.g. as disclosed above,
each being releasably affixed to the delivery device or system.
2.2 A device as defined herein for use in any method as defined under 1.1 to 1.12.
3.1 Use of rapamycin or a rapamycin derivative having mTOR inhibiting properties in any of the method as defined under 1.4, 1.6 or 1.9 optionally in conjunction with one or more other active co-agent, or in the manufacture of a medicament for use in any of the method as defined under 1.4, 1.6 or 1.9 optionally in conjunction with one or more other active co-agent.
3.2 Use of a rapamycin derivative having mTOR inhibiting properties, optionally in combination with an active co-agent as defined herein, in the manufacture of a device as defined herein for use in any method as defined under 1.1 to 1.12.
3.3 Use of indwelling shunt, fistula or catheter coated by, impregnated with or incorporating rapamycin or a rapamycin derivative having mTOR inhibiting properties (i.e. being releasably affixed to the medical device) as described herein, for the manufacture of a medicament for the prevention or reduction of vascular access dysfunction in association with the insertion or repair of an indwelling shunt, fistula or catheter into a vein or artery, in a subject in need thereof.
4. A pharmaceutical composition for use in any method as defined under 1.4, 1.6 or 1.9 comprising rapamycin or a derivative thereof having mTOR properties, e.g. CC1779, ABT578, a rapalog or a compound of formula I, together with one or more pharmaceutically acceptable diluents or carriers therefor.

A local delivery device or system according to the invention can be used to reduce stenosis or restenosis as an adjunct to revascularization, bypass or grafting procedures performed in any vascular location including coronary arteries, carotid arteries, renal arteries, peripheral arteries, cerebral arteries or any other arterial or venous location, to reduce anastomic stenosis or hyperplasia including in the case of arterial-venous dialysis access with or without PTFE or e.g. Gore-Tex grafting and with or without stenting, or in conjunction with any other heart or transplantation procedures, or congenital vascular interventions.

In a preferred embodiment, the present invention also provides a drug delivery system or device as disclosed above additionally comprising a source delivering a therapeutic dosage of a compound or antibody which inhibits the PDGF receptor tyrosine kinase or a compound which binds to PDGF or reduces expression of the PDGF receptor e.g. as disclosed above, a compound or antibody which inhibits the EGF receptor tyrosine kinase or a compound which binds to EGF or reduces expression of the EGF receptor e.g. as disclosed above, a compound or antibody which inhibits the VEGF receptor tyrosine kinase or a VEGF receptor or a compound which binds to VEGF, e.g. as disclosed above, each being releasably affixed to the catheter-based delivery device or medical device.

Rapamycin or rapamycin derivative having mTOR inhibiting properties will be referred to hereinafter as “active agent”. “Drug(s)” means active agent or the active agent and the active co-agent.

The local administration preferably takes place at or near the lesion sites, e.g. vascular lesion sites.

The local administration may be by one or more of the following routes: via catheter or other intravascular delivery system, intranasally, intrabronchially, interperitoneally or eosophagal, or via delivery balloons used in the musculature. Hollow tubes include natural body vessels or ducts, e.g. circulatory system vessels such as blood vessels (arteries or veins), tissue lumen, lymphatic pathways, digestive tract including alimentary duct, e.g. esophagus or biliary ducts, respiratory tract, e.g. trachea, excretory system tubes, e.g. intestines, ureters or urethra-prostate, reproductive system tubes and ducts, body cavity tubes, etc. Local administration or application of the drug(s) may afford concentrated delivery of said drug(s), achieving tissue levels in target tissues not otherwise obtainable through other administration route. Additionally local administration or application may reduce the risk of remote or systemic toxicity. Preferably the smooth muscle cell proliferation or migration is inhibited or reduced according to the invention immediately proximal or distal to the locally treated or stented area.

Means for local delivery of the drug(s) to hollow tubes can be by physical delivery of the drug(s) either internally or externally to the hollow tube. Local drug(s) delivery includes catheter delivery systems, local injection devices or systems or indwelling devices. Such devices or systems would include, but not be limited to, stents, coated stents, endolumenal sleeves, stent-grafts, sheathes, balloons, liposomes, controlled release matrices, polymeric endoluminal paving, or other endovascular devices, embolic delivery particles, cell targeting such as affinity based delivery, internal patches around the hollow tube, external patches around the hollow tube, hollow tube cuff, external paving, external stent sleeves, and the like. See, Eccleston et al. (1995) Interventional Cardiology Monitor 1:33-40-41, Slepian, N.J. (1996) Interventional Cardiol. 1:103-116, or Regar E, Sianos G, Serruys P W, Stent development and local drug delivery, Br Med Bull 2001, 59:227-48, which disclosures are herein incorporated by reference.

Preferably the delivery device or system fulfils pharmacological, pharmacokinetic and mechanical requirements. Preferably it also is suitable for sterilisation.

The stent according to the invention can be any stent, including self-expanding stent, or a stent that is radially expandable by inflating a balloon or expanded by an expansion member, or a stent that is expanded by the use of radio frequency which provides heat to cause the stent to change its size. A stent composed of or coated with a polymer or other biocompatible materials, e.g. porous ceramic, e.g. nanoporous ceramic, into which the drug(s) has been impregnated or incorporated can be used. Stents can be biodegradable or can be made of metal or alloy, e.g. Ni and Ti, or another stable substance when intented for permanent use. The drug(s) may also be entrapped into the metal of the stent or graft body which has been modified to contain micropores or channels. Also lumenal and/or ablumenal coating or external sleeve made of polymer or other biocompatible materials, e.g. as disclosed below, that contain the drug(s) can also be used for local delivery.

By “biocompatible” is meant a material which elicits no or minimal negative tissue reaction including e.g. thrombus formation and/or inflammation.

Stents may commonly be used as a tubular structure left inside the lumen of a duct to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to shear and disrupt the obstructions associated with the wall components of the vessel and to obtain an enlarged lumen. Alternatively, stents being easily deformed at lower temperature to be inserted in the hollow tubes may be used: after deployment at site, such stents recover their original shape and exert a retentive and gentle force on the internal wall of the hollow tubes, e.g. of the esophagus or trachea.

The drug(s) may be incorporated into or affixed to the stent in a number of ways and utilizing any biocompatible materials; it may be incorporated into e.g. a polymer or a polymeric matrix and sprayed onto the outer surface of the stent. A mixture of the drug(s) and the polymeric material may be prepared in a solvent or a mixture of solvents and applied to the surfaces of the stents also by dip-coating, brush coating and/or dip/spin coating, the solvent (s) being allowed to evaporate to leave a film with entrapped drug(s). In the case of stents where the drug(s) is delivered from micropores, struts or channels, a solution of a polymer may additionally be applied as an outlayer to control the drug(s) release; alternatively, the active agent may be comprised in the micropores, struts or channels and the active co-agent may be incorporated in the outlayer, or vice versa. The active agent may also be affixed in an inner layer of the stent and the active co-agent in an outer layer, or vice versa. The drug(s) may also be attached by a covalent bond, e.g. esters, amides or anhydrides, to the stent surface, involving chemical derivatization. The drug(s) may also be incorporated into a biocompatible porous ceramic coating, e.g. a nanoporous ceramic coating. The medical device of the invention is configured to release the active co-agent concurrent with or subsequent to the release of the active agent.

Examples of polymeric materials include hydrophilic, hydrophobic or biocompatible biodegradable materials, e.g. polycarboxylic acids; cellulosic polymers; starch; collagen; hyaluronic acid; gelatin; lactone-based polyesters or copolyesters, e.g. polylactide; polyglycolide; polylactide-glycolide; polycaprolactone; polycaprolactone-glycolide; poly(hydroxybutyrate); poly(hydroxyvalerate); polyhydroxy(butyrate-co-valerate); polyglycolide-co-trimethylene carbonate; poly(diaxanone); polyorthoesters; polyanhydrides; polyaminoacids; polysaccharides; polyphospoeters; polyphosphoester-urethane; polycyanoacrylates; polyphosphazenes; poly(ether-ester) copolymers, e.g. PEO-PLLA, fibrin; fibrinogen; or mixtures thereof; and biocompatible non-degrading materials, e.g. polyurethane; polyolefins; polyesters; polyamides; polycaprolactame; polyimide; polyvinyl chloride; polyvinyl methyl ether; polyvinyl alcohol or vinyl alcohol/olefin copolymers, e.g. vinyl alcohol/ethylene copolymers; polyacrylonitrile; polystyrene copolymers of vinyl monomers with olefins, e.g. styrene acrylonitrile copolymers, ethylene methyl methacrylate copolymers; polydimethylsiloxane; poly(ethylene-vinylacetate); acrylate based polymers or coplymers, e.g. polybutylmethacrylate, poly(hydroxyethyl methylmethacrylate); polyvinyl pyrrolidinone; fluorinated polymers such as polytetrafluoethylene; cellulose esters e.g. cellulose acetate, cellulose nitrate or cellulose propionate; or mixtures thereof.

When a polymeric matrix is used, it may comprise 2 layers, e.g. a base layer in which the drug(s) is/are incorporated, e.g. ethylene-co-vinylacetate and polybutylmethacrylate, and a top coat, e.g. polybutylmethacrylate, which is drug(s)-free and acts as a diffusion-control of the drug(s). Alternatively, the active agent may be comprised in the base layer and the active co-agent may be incorporated in the outlayer, or vice versa. Total thickness of the polymeric matrix may be from about 1 to 20μ or greater.

According to the method of the invention or in the device or system of the invention, the drug(s) may elute passively, actively or under activation, e.g. light-activation.

The drug(s) elutes from the polymeric material or the stent over time and enters the surrounding tissue, e.g. up to ca. 1 month to 1 year. The local delivery according to the present invention allows for high concentration of the drug(s) at the disease site with low concentration of circulating compound. The amount of drug(s) used for local delivery applications will vary depending on the compounds used, the condition to be treated and the desired effect. For purposes of the invention, a therapeutically effective amount will be administered; for example, the drug delivery device or system is configured to release the active agent and/or the active co-agent at a rate of 0.001 to 200 μg/day. By therapeutically effective amount is intended an amount sufficient to inhibit cellular proliferation and resulting in the prevention and treatment of the disease state. Specifically, for the prevention or treatment of restenosis e.g. after revascularization, or antitumor treatment, local delivery may require less compound than systemic administration.

A contemplated treatment period for use in the prevention or reduction of vascular access dysfunction of the present invention is about 85, e.g. 70, preferably 50, e.g. 28, more preferably 28 days in association with the insertion or repair of an indwelling shunt, fistula or catheter, or actual treatment.

A preferred method of use in the prevention or reduction of vascular access dysfunction is a method for preventing or reducing vascular thrombosis and/or fistula failure and/or shunt failure and/or vascular access clotting and/or stenosis and/or restenosis and/or the need for declotting an indwelling access clotting shunt, fistula or catheter associated with insertion or repair of the indwelling shunt, fistula or catheter, or actual treatment, in dialysis patients.

A preferred method of use in the prevention or reduction of vascular access dysfunction is a method for preventing or reducing vascular thrombosis and/or fistula failure and/or shunt failure and/or vascular access clotting and/or stenosis and/or restenosis and/or the need for declotting an indwelling vascular access shunt, fistula or catheter associated with insertion or repair of the indwelling shunt, fistula or catheter, or actual treatment, in cancer patients.

A preferred method of use in the prevention or reduction of vascular access dysfunction is a method for preventing or reducing vascular thrombosis and/or fistula failure and/or shunt failure and/or vascular access clotting and/or stenosis and/or restenosis and/or the need for declotting an indwelling vascular access shunt, fistula or catheter associated with insertion or repair of the indwelling shunt, fistula or catheter, or actual treatment, in total parenteral nutrition (TPN) patients.

By “prevention or reduction of vascular access dysfunction in association with the insertion or repair of an indwelling shunt, fistula or catheter” as used herein, is meant that the incidence of vascular thrombosis and/or fistula failure and/or shunt failure and/or vascular access clotting and/or stenosis and/or restenosis and/or the need for declotting an indwelling vascular access shunt, fistula or catheter in patients treated according to the invention collected over the observation period are prevented or reduced in comparison to untreated patients.

By “in association with the insertion or repair of an indwelling shunt, fistula or catheter” as used herein, is meant that the treatment according to the invention can commence immediately, for example within 4 to 8 hours, after insertion or repair of the indwelling shunt, fistula or catheter, or actual treatment, such as dialysis treatment; within a few days, for example about 7 days, preferably about 1 or 2 days, after insertion or repair of the indwelling shunt, fistula or catheter, or actual treatment, such as dialysis treatment; or for a period of days, for example about 30 days, preferably about 14 days, preferably about 7 days, prior to insertion or repair of the indwelling shunt, fistula or catheter, or actual treatment, such as dialysis treatment. Also contemplated within the phrase “in association with the insertion or repair of an indwelling shunt, fistula or catheter” is a dosing protocol in which a dose or several doses, are skipped, for example in the morning of or on the day of insertion, repair or treatment. Also contemplated within the phrase “in association with the insertion or repair of an indwelling shunt, fistula or catheter” is a dosing protocol in which a day of drug treatment or several days of drug treatment, are skipped.

Included in term “treatment”, when used herein to refer surgical procedures, are procedures selected from access surgery, placement of fistula or shunt, catheter insertion, actual disease treatment, such as dialysis treatment, and declotting of an access shunt, fistula or catheter. Further, treatment for insertion access also includes repair/revision of the access. For example, a patient experiencing a failure in a dialysis access shunt will have the access repaired, for instance, by angioplasty.

By the term “collected over the observation period” as used herein, means a period of up to or about 12 months, preferably 12 months.

When rapamycin or a rapamycin derivative having mTOR inhibiting properties is administered systemically or is additionally administered by systemic application, e.g. in the prevention or reduction of vascular access dysfunction, according to the invention, daily dosages required in practicing the method of the present invention will vary depending upon, for example, the compound used, the host, the mode of administration and the severity of the condition to be treated. A preferred daily dosage range is about from 0.1 to 25 mg as a single dose or in divided doses. Suitable daily dosages for patients are on the order of from e.g. 0.1 to 25 mg p.o. The compound may be administered by any conventional route, in particular enterally, e.g. orally, e.g. in the form of tablets, capsules, drink solutions, nasally, pulmonary (by inhalation) or parenterally, e.g. in the form of injectable solutions or suspensions. Suitable unit dosage forms for oral administration comprise from ca. 0.05 to 12.5 mg, usually 0.25 to 10 mg compound, together with one or more pharmaceutically acceptable diluents or carriers therefor.

Preferred combinations according to the invention are those comprising a compound of formula I, e.g. 40-O-(2-hydroxyethyl)-rapamycin or 32-deoxorapamycin, or CCI-779, ABT578 or a rapalog in conjunction or association with a compound having antiproliferative properties, e.g. taxol, paclitaxel, docetaxel, an epothilone, a tyrosine kinase inhibitor, e.g. a protein kinase C or PI(3) kinase inhibitor, for example staurosporin or a related small molecule, a PDGF receptor tyrosine kinase inhibitor, a PDGF receptor inhibitor, a compound binding to PDGF, e.g. imatinib, a VEGF receptor tyrosine kinase inhibitor, a VEGF receptor inhibitor, a compound binding to VEGF, e.g. 1-(4-chloroanilino)-4-(4-pyridylmethyl)phtalazine, a cox-2 inhibitor, an ascomycin, e.g. pimecrolimus, or a calcineurin inhibitor, e.g. CysA, ISA tx 247 or FK506. A combination of rapamycin or a rapamycin derivative as mentioned above with a compound having anti-inflammatory properties, pimecrolimus, or an EDG-receptor agonist having lymphocyte depleting properties, has particularly beneficial effects when used in the treatment or prevention of restenosis in diabetic patients. A combination of rapamycin or a rapamycin derivative as mentioned above with a statin or an aldosterone synthetase inhibitor or an aldosterone receptor blocker, or with a compound inhibiting the renin-angiotensin system has also beneficial properties; such a combination also forms part of the invention.

Rapamycin or the rapamycin derivative having mTOR inhibiting properties may also be applied to the drug delivery device or system in admixture with an antioxidant, e.g. 2,6-di-tert.-butyl-4-methylphenol, e.g. at an amount up to 0.5% by weight, preferably 0.2% by weight.

Utility of the drug(s) may be demonstrated in animal test methods as well as in clinic, for example in accordance with the methods hereinafter described.

A1. Inhibition of Late Neointimal Lesion Formation in the 28 Day Rat Carotid Artery Balloon Injury Model

Numerous compounds have been shown to inhibit intimal lesion formation at 2 weeks in the rat ballooned carotid model, while only few compounds prove effective at 4 weeks. Compounds of formula I are tested in the following rat model.

Rats are dosed orally with placebo or a compound of formula I. Daily dosing starts 3 days prior to surgery and continues for 31 days. Rat carotid arteries are balloon injured using a method described by Clowes et al. Lab. Invest. 1983; 49; 208-215. Following sacrifice at 28 days post-balloon injury, carotid arteries are removed and processed for histologic and morphometric evaluation. In this assay the compounds of formula I, e.g. 40-O-(2-hydroxyethyl)-rapamycin, significantly reduce neointimal lesion formation at 28 days following balloon injury when administered at a dose of from 0.5 to 2.0 mg/kg. For example for 40-O-(2-hydroxyethyl)-rapamycin administered at 0.5, 1.0, and 2.0 mg/kg, the percent inhibition is similar at all three doses: inhibition is 31% at the lowest dose (0.5 mg/kg) and 39% at the highest dose (2.0 mg/kg). Compounds of formula I, e.g. 40-O-(2-hydroxyethyl)-rapamycin, have the beneficial effect to inhibit lesions at 4 weeks post-ballooning.

A.2 Inhibition of Restenosis at 28 Days in the Rabbit Iliac Stent Model

A combined angioplasty and stenting procedure is performed in New Zealand White rabbit iliac arteries. Iliac artery balloon injury is performed by inflating a 3.0×9.0 mm angioplasty balloon in the mid-portion of the artery followed by “pull-back” of the catheter for 1 balloon length. Balloon injury is repeated 2 times, and a 3.0×12 mm stent is deployed at 6 atm for 30 seconds in the iliac artery. Balloon injury and stent placement is then performed on the contralateral iliac artery in the same manner. A post-stent deployment angiogram is performed. All animals receive oral aspirin 40 mg/day daily as anti-platelet therapy and are fed standard low-cholesterol rabbit chow. Twenty-eight days after stenting, animals are anesthetized and euthanized and the arterial tree is perfused at 100 mmHg with lactated Ringer's for several minutes, then perfused with 10% formalin at 100 mmHg for 15 minutes. The vascular section between the distal aorta and the proximal femoral arteries is excised and cleaned of periadventitial tissue. The stented section of artery is embedded in plastic and sections are taken from the proximal, middle, and distal portions of each stent. All sections are stained with hematoxylin-eosin and Movat pentachrome stains. Computerized planimetry is performed to determine the area of the internal elastic lamina (IEL), external elastic lamina (EEL) and lumen. The neointima and neointimal thickness is measured both at and between the stent struts. The vessel area is measured as the area within the EEL. Data are expressed as mean±SEM. Statistical analysis of the histologic data is accomplished using analysis of variance (ANOVA) due to the fact that two stented arteries are measured per animal with a mean generated per animal. A P<0.05 is considered statistically significant.

A compound of formula I, e.g. 40-O-(2-hydroxyethyl)-rapamycin, is administered orally by gavage at a loading dose of 1.5 mg/kg one day prior to stenting, then dosed at 0.75 mg/kg/day from the day of stenting until day 27 post-stenting. In this model, the treatment with the compounds of formula I results in a marked reduction in the extent of restenotic lesion formation: for example, the treatment with 40-O-(2-hydroxyethyl)-rapamycin produces a significant (P<0.03) reduction in neointimal thickness (40% reduction), neointimal area (24% reduction), and percent arterial stenosis (26% reduction) with a significant 32% increase in lumen area. There is extensive neointimal formation in placebo-treated animals at 28 days, with the lesions consisting of abundant smooth muscle cells in proteoglycan/collagen matrix and apparent full endothelial healing. In the majority of arterial segments from the animals treated with 40-O-(2-hydroxyethyl)-rapamycin, the intima is well healed, characterized by a compact neointimal consisting of smooth muscle cells and endothelium both over stent struts and between struts. Scanning electron microscopic analysis shows that stented arteries from the animals treated with 40-O-(2-hydroxyethyl)-rapamycin (n=4 arteries) was 84% endothelialized.

A.3 Inhibition of Restenosis at 14 Days in the Rat Carotid Stent Model

Male Sprague Dawley rats weighing 250 to 500 mg are housed individually and allowed to acclimate prior to surgery. All animals receive standard rat chow and water ad libitum. Group size is 12 animals per group.

The drug(s) administration is perivascular. A segment of ballooned carotid is encircled with a 1 cm length of silastic tubing (0.25 inch inside diameter, 0.47 inch outside diameter) to which is attached a catheter which feeds into an osmotic pump containing either compound or vehicle. This delivery system provides continuous, local delivery to the adventitia of the wrapped portion of vessel. Local drug(s) administration ranges between 5 μg and 10 mg, locally per day, depending on the solubility characteristics of the individual compounds.

The left common carotid arteries are denuded of endothelium using a 2F Fogarty catheter as previously described (Prescott Am. J. Pathol. (1991) 139:1291-1296, Clowes et al., (1983) Lab Invest. 49:327-333). Briefly, rats are anesthetized with ketamine (50 mg/ml) and rompun (10 mg/ml) administered intraperitoneally at a dose of 1.5 ml/kg. A midline incision is made in the neck to expose the left external and common carotid arteries. The balloon is inserted into the common carotid artery via the left external branch, inflated with saline, and pulled back three times through the lumen with a rotating motion to ensure maximal endothelial denudation. The catheter is then removed, the external carotid artery is ligated and the wound is closed. Each animal is given an injection of the antibiotic Bacillin (200.000 units/kg) and the analgesic Buprenophine (0.06 mg/kg) immediately following surgery.

Animals are killed at 14 days post-balloon injury. One half hour before termination blood is collected, centrifuged, and stored at −20° C. for analysis of circulating levels of compound. 5% Evans Blue is then injected intravenously to allow discrimination of re-endothelialized areas at the time of histologic processing. Animals are killed by administration of an overdose of ketamine and rompun, the osmotic pumps are recovered and the volume of remaining content is recorded to ensure that pump failure has not occurred.

Carotid arteries are excised and immersion fixed, then transferred to Ringer's solution. Two samples from control blue region of each left carotid artery are imbedded in paraffin. A minimum of six carotid sections, 20 μM apart are cut per animal and stained with Verhoff Elastic stain to produce a modified Verhoff stain. Intimal and medial area measurements are performed with a computerized imaging system. The intimal lesion area and the medial area are determined by measurement of the internal elastic lamina, the external elastic lamina and the vessel/lumen interface.

In this assay, 40-O-(2-hydroxyethyl)-rapamycin reduces neointimal lesion formation at 14 days post ballooning when administered locally as disclosed above at a dose of 10 to 200 μg/day. Similar good results are obtained when 40-O-(2-hydroxyethyl)-rapamycin is administered in conjunction with dexamethasone (10-250 μg/day) or a tyrosine kinase inhibitor or an anti-inflammatory agent, e.g. pimecrolimus.

A4. Treatment of Angina Pectoris Patients

25 patients with angina pectoris are treated with a stent according to the invention, e.g. delivering a rapamycin derivative having mTOR inhibiting properties. The stents (15 mm) are delivered to the patients (3.0-3.5 mm vessel calibre) and the patients are discharged without clinical complications. At 4 months and 1 year angiographic and IVUS follow-up, no significant neo-intimal hyperplasia is detected.

In this trial, when a stent delivering rapamycin or a derivative thereof having mTOR inhibiting properties in conjunction with pimecrolimus or midostaurin is used, beneficials effects are obtained.

A5. Prevention or Reduction of Vascular Access Dysfunction in Association with the Insertion of an Indwelling Catheter into the Vein of a Patient

One hundred fifty prospective dialysis patients, who undergo successful insertion of an indwelling, large bore catheter, into a vein are selected for the study. These patients are divided into two groups, and both groups do not differ significantly with sex, distribution of vascular condition or condition of lesions after insertion. One group (about 50 patients) receives rapamycin or a rapamycin derivative having mTOR inhibiting properties in a daily dose of 0.75 to 20 mg (hereinafter identified as group 1), and another group (about 100 patients) does not receive the compound to be tested (hereinafter identified as group H). In addition, patients may also be given a calcium antagonist, nitrates and/or anti-platelet agents. These drugs are administered for 3 consecutive months following catheter insertion. The comparative clinical data collected over the observation period of 6 months demonstrate the efficacy of 3 month treatment with rapamycin or a rapamycin derivative, e.g. 40-O-(2-hydroxyethyl)-rapamycin, for the prevention or reduction of vascular access dysfunction in patients after catheter insertion.

The following examples are illustrative of the invention without limitating it.

### EXAMPLE 1

The stent is manufactered from medical 316LS stainless steel and is composed of a series of cylindrically oriented rings aligned along a common longitudinal axis. Each ring consists of 3 connecting bars and 6 expanding elements. The stent is premounted on a delivery system. The active agent, e.g. 40-(2-hydroxyethyl)-rapamycin (0.50 mg/ml) optionally together with 2,6-di-tert.-butyl-4-methylphenol (0.001 mg/ml), is incorporated into a polymer matrix based on a semicrystalline ethylene-vinyl alcohol copolymer. The stent is coated with this matrix.

### EXAMPLE 2

A stent is weighed and then mounted for coating. While the stent is rotating, a solution of polylactide glycolide, 0.75 mg/ml of 40-O-(2-hydroxyethyl)-rapamycin, 0.0015 mg/ml 2,6-di-tert.-butyl-4-methylphenol and 1 mg/ml tyrosine kinase C inhibitor dissolved in a mixture of methanol and tetrahydrofuran, is sprayed onto it. The coated stent is removed from the spray and allowed to air-dry. After a final weighing the amount of coating on the stent is determined.

The tyrosine kinase inhibitor C may be replaced by taxol, paclitaxel, a VEGF receptor tyrosine kinase inhibitor, a VEGF receptor inhibitor, a compound binding to VEGF, an aldosterone synthetase inhibitor or an aldosterone receptor blocker, or a compound inhibiting the renin-angiotensin system.

### EXAMPLE 3

Four 2 cm pieces of coated stents as described above are placed into 100 ml of phosphate buffer solution (PBS) having a pH of 7.4. Another 4 pieces from each series are placed into 100 ml polyethylene glycol (PEG)/water solution (40/60 v/v, MW of PEG=400). The stent pieces are incubated at 37° C. in a shaker. The buffer and PEG solutions are changed daily and different assays are performed on the solution to determine the released 40-O-(2-hydroxyethyl)-rapamycin concentrations. Such assays can show a stable release of 40-O-(2-hydroxyethyl)-rapamycin from coated stents for more than 45 days. By the term “stable release of 40-O-(2-hydroxyethyl)-rapamycin” is meant less than 10% of variation of the drug release. Controlled release techniques used by a person skilled in the art allow an unexpected easy adaptation of the required drug release rate. Thus, by selecting appropriate amounts of reactants in the coating mixture it is possible to easily control the bioeffectiveness of the rapamycin or rapamycin derivastive coated stents.

1. A drug delivery device or system comprising i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from an EDG-receptor agonist having lymphocyte depleting properties, a cox-2 inhibitor, pimecrolimus, a cytokine inhibitor, a chemokine inhibitor, an antiproliferative agent, a statin, a protein, growth factor or compound stimulating growth factor production that will enhance endothelial regrowth of the luminal endothelium, a matrix metalloproteinase inhibitor, a somatostatin analogue, an aldosterone synthetase inhibitor or aldosterone receptor blocker and a compound inhibiting the renin-angiotensin system, each being releasably affixed to the medical device.

2. A drug delivery device or system comprising i) a medical device adapted for local application or administration of an active ingredient in hollow tubes and ii) an active ingredient comprising a therapeutic dosage of a rapamycin derivative having mTOR inhibiting properties, in conjunction with a therapeutic dosage of one or more active co-agents selected from a calcineurin inhibitor and mycophenolic acid or a salt thereof or prodrug thereof, each being releasably affixed to the medical device.

3. The drug delivery device or system according to claim 1 wherein the medical device is a stent or coated stent.

4. The drug delivery device or system according to claim 2 wherein the medical device is a stent or coated stent.

5. A combination of rapamycin or a rapamycin derivative having mTOR inhibiting properties with pimecrolimus, an aldosterone synthetase inhibitor or an aldosterone receptor blocker, or with a compound inhibiting the renin-angiotensin system.

6. A method for preventing or treating smooth muscle cell proliferation and migration in hollow tubes, or increased cell proliferation or decreased apoptosis or increased matrix deposition in a subject in need thereof, comprising local administration of a therapeutically effective amount of an active ingredient comprising a rapamycin derivative having mTOR inhibiting properties or rapamycin in conjunction with one or more active co-agents selected from an EDG-receptor agonist having lymphocyte depleting properties; a cox-2 inhibitor; pimecrolimus; a cytokine inhibitor; a chemokine inhibitor; an antiproliferative agent; a statin, a protein, growth factor or compound stimulating growth factor production that will enhance endothelial regrowth of the luminal endothelium; a matrix metalloproteinase inhibitor; a somatostatin analogue; an aldosterone synthetase inhibitor or aldosterone receptor blocker; and a compound inhibiting the renin-angiotensin system.

7. A method according to claim 6 wherein the local administration is the controlled delivery from a drug delivery device or system comprising i) a medical device adapted for local application or administration of an active ingredient in hollow tubes and ii) the active ingredient wherein the active ingredient is releasably affixed to the medical device.

8. A method according to claim 6 wherein the local administration is the controlled delivery from a drug delivery device or system comprising i) a medical device adapted for local application or administration of an active ingredient in hollow tubes and ii) the active ingredient wherein the active ingredient comprises a therapeutic dosage of a rapamycin derivative having mTOR inhibiting properties, in conjunction with a therapeutic dosage of one or more active co-agents selected from a calcineurin inhibitor and mycophenolic acid or a salt thereof or prodrug thereof, each being releasably affixed to the medical device.

9. A method for stabilizing vulnerable plaques in blood vessels of a subject in need of such a stabilization comprising administering to said patient a therapeutically effective amount of rapamycin or a rapamycin derivative having mTOR inhibiting properties, optionally in conjunction with one or more active co-agents and/or a controlled delivery from a drug delivery device or system of a therapeutically effective amount of rapamycin or a rapamycin derivative having mTOR inhibiting properties, optionally in conjunction with one or more active co-agents.

10. A method according to claim 9 wherein the drug delivery device or system comprises i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from a different immunosuppressant; an EDG-receptor agonist having lymphocyte depleting properties; an anti-inflammatory agent; an anti-thrombotic or anti-coagulant agent; an antiproliferative agent; a statin; a protein, growth factor or compound stimulating growth factor production that will enhance endothelial regrowth of the luminal endothelium; a matrix metalloproteinase inhibitor; a modulator of kinases; a compound stimulating the release of NO or a NO donor; a somatostatin analogue; an aldosterone synthetase inhibitor or aldosterone receptor blocker; a compound inhibiting the renin-angiotensin system; and mycophenolic acid or salt or prodrug thereof; each being releasably affixed to the medical device.

11. A method according to claim 9 wherein the drug delivery device or system comprises i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from an EDG-receptor agonist having lymphocyte depleting properties; a cox-2 inhibitor; pimecrolimus; a cytokine inhibitor; a chemokine inhibitor; an antiproliferative agent; a statin, a protein, growth factor or compound stimulating growth factor production that will enhance endothelial regrowth of the luminal endothelium; a matrix metalloproteinase inhibitor; a somatostatin analogue; an aldosterone synthetase inhibitor or aldosterone receptor blocker; and a compound inhibiting the renin-angiotensin system; each being releasably affixed to the medical device.

12. A method according to claim 9 wherein the drug delivery device or system comprises i) a medical device adapted for local application or administration of an active ingredient in hollow tubes and and ii) an active ingredient comprising a therapeutic dosage of a rapamycin derivative having mTOR inhibiting properties, in conjunction with a therapeutic dosage of one or more active co-agents selected from a calcineurin inhibitor and mycophenolic acid or a salt thereof or prodrug thereof, each being releasably affixed to the medical device.

13. A method for preventing or treating restenosis in a diabetic patient comprising administering to said patient a therapeutically effective amount of rapamycin or a rapamycin derivative having mTOR inhibiting properties, optionally in conjunction with one or more active co-agents, and/or a controlled delivery from a drug delivery device or system of a therapeutically effective amount of rapamycin or a rapamycin derivative having mTOR inhibiting properties, optionally in conjunction with one or more active co-agents.

14. A method according to claim 13 comprising the controlled delivery from a drug delivery device or system wherein the drug delivery device or system comprises i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from a different immunosuppressant; an EDG-receptor agonist having lymphocyte depleting properties; an anti-inflammatory agent; an anti-thrombotic or anti-coagulant agent; an antiproliferative agent; a statin; a protein, growth factor or compound stimulating growth factor production that will enhance endothelial regrowth of the luminal endothelium; a matrix metalloproteinase inhibitor; a modulator of kinases; a compound stimulating the release of NO or a NO donor; a somatostatin analogue; an aldosterone synthetase inhibitor or aldosterone receptor blocker; a compound inhibiting the renin-angiotensin system; and mycophenolic acid or salt or prodrug thereof; each being releasably affixed to the medical device.

15. A method according to claim 13 comprising the controlled delivery from a drug delivery device or system wherein the drug delivery device or system comprises i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from an EDG-receptor agonist having lymphocyte depleting properties; a cox-2 inhibitor; pimecrolimus; a cytokine inhibitor; a chemokine inhibitor; an antiproliferative agent; a statin; a protein, growth factor or compound stimulating growth factor production that will enhance endothelial regrowth of the luminal endothelium; a matrix metalloproteinase inhibitor; a somatostatin analogue; an aldosterone synthetase inhibitor or aldosterone receptor blocker; and a compound inhibiting the renin-angiotensin system; each being releasably affixed to the medical device

16. A method according to claim 13 comprising the controlled delivery from a drug delivery device or system wherein sain drug delivery device or system comprises i) a medical device adapted for local application or administration of an active ingredient in hollow tubes and and ii) an active ingredient comprising a therapeutic dosage of a rapamycin derivative having mTOR inhibiting properties, in conjunction with a therapeutic dosage of one or more active co-agents selected from a calcineurin inhibitor and mycophenolic acid or a salt thereof or prodrug thereof, each being releasably affixed to the medical device.

17. A method for the prevention or reduction of vascular access dysfunction in association with the insertion or repair of an indwelling shunt, fistula or catheter, or actual treatment, in a subject in need thereof, which comprises administering to the subject rapamycin or a rapamycin derivative having mTOR inhibiting properties, optionally in conjunction with one or more active co-agents, and/or a controlled delivery from a drug delivery device or system of a therapeutically effective amount of rapamycin or a rapamycin derivative having mTOR inhibiting properties, optionally in conjunction with one or more other active co-agents.

18. A method according to claim 17 comprising the controlled delivery from a drug delivery device or system wherein the drug delivery device or system comprises i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from a different immunosuppressant; an EDG-receptor agonist having lymphocyte depleting properties; an anti-inflammatory agent; an anti-thrombotic or anti-coagulant agent; an antiproliferative agent; a statin; a protein, growth factor or compound stimulating growth factor production that will enhance endothelial regrowth of the luminal endothelium; a matrix metalloproteinase inhibitor; a modulator of kinases; a compound stimulating the release of NO or a NO donor; a somatostatin analogue; an aldosterone synthetase inhibitor or aldosterone receptor blocker; a compound inhibiting the renin-angiotensin system; and mycophenolic acid or salt or prodrug thereof; each being releasably affixed to the medical device.

19. A method according to claim 17 comprising the controlled delivery from a drug delivery device or system wherein the drug delivery device or system comprises i) a medical device adapted for local application or administration of an active ingredient in hollow tubes and ii) an active ingredient comprising a therapeutic dosage of a rapamycin derivative having mTOR inhibiting properties or rapamycin, in conjunction with a therapeutic dosage of one or more active co-agents selected from an EDG-receptor agonist having lymphocyte depleting properties; a cox-2 inhibitor; pimecrolimus; a cytokine inhibitor; a chemokine inhibitor; an antiproliferative agent; a statin, a protein, growth factor or compound stimulating growth factor production that will enhance endothelial regrowth of the luminal endothelium; a matrix metalloproteinase inhibitor; a somatostatin analogue; an aldosterone synthetase inhibitor or aldosterone receptor blocker; and a compound inhibiting the renin-angiotensin system; each being releasably affixed to the medical device

20. A method according to claim 17 comprising the controlled delivery from a drug delivery device or system wherein sain drug delivery device or system comprises i) a medical device adapted for local application or administration of an active ingredient in hollow tubes and and ii) an active ingredient comprising a therapeutic dosage of a rapamycin derivative having mTOR inhibiting properties, in conjunction with a therapeutic dosage of one or more active co-agents selected from a calcineurin inhibitor and mycophenolic acid or a salt thereof or prodrug thereof, each being releasably affixed to the medical device.
